(19) United States
(12) Patent Application Publication
(10) Pub. No.: US 2024/0003850 A1
(43) Pub. Date: Jan. 4, 2024

(54) REAL-TIME CONDITION ASSESSMENT OF LIVING PLANTS BY DISTRIBUTED SENSING OF PLANT-EMITTED VOLATILES

(71) Applicant: NORTH CAROLINA STATE UNIVERSITY, Raleigh, NC (US)

(72) Inventors: Omer Oralkan, Morrisville, NC (US); Ralph Adrian Dean, Raleigh, NC (US); Fatih Erden, Cary, NC (US); Nasie Constantino, Cary, NC (US); Yeonyee Oh, Cary, NC (US); Erdem Sennik, Raleigh, NC (US)

(21) Appl. No.: 18/253,577

(22) PCT Filed: Dec. 1, 2021
(86) PCT No.: PCT/US21/61432

§ 371 Date: May 18, 2023

Related U.S. Application Data
(60) Provisional application No. 63/120,233, filed on Dec. 2, 2020.

(51) Int. Cl. G01N 29/028; A01G 9/26; G01N 29/24; G01N 29/44; G01N 33/00

(52) U.S. Cl. CPC G01N 29/028; A01G 9/26; G01N 29/2406; G01N 29/4481; G01N 33/0098; G01N 2291/018; G01N 2291/02809

(57) ABSTRACT

Aspects and features of this disclosure include a gas sensing platform with small, low-power, wireless gas sensor packages for selective detection of VOCs released from plants under different conditions, including abiotic or biotic stress conditions. The sensor packages for the platform can be implemented using an array of capacitive micromachined ultrasonic transducer (CMUT) arrays, in which elements are functionalized with a variety of materials. A computing platform can receive data from the arrays of sensors. The computing platform can determine a characteristic about a nearby plant or nearby plants based on chemicals detected in the gas emissions from the plants and produce a plant condition assessment based on the characteristic.

### CROSS-REFERENCE TO RELATED APPLICATION

This claims priority to U.S. Provisional Patent Application 63/120,233 filed Dec. 2, 2020, the entire disclosure of which is incorporated herein by reference.

### TECHNICAL FIELD

This disclosure generally relates to sensors and systems for use with living plants. More specifically, but not by way of limitation, this disclosure relates to sensors and systems that can provide a condition assessment of the plants.

### BACKGROUND

During times of stress, possibly due to herbivory or pathogen infection, plants emit a wide range of volatile organic compounds (VOCs). These VOCs serve to activate the plant defenses, attract beneficial insects, and warn adjacent plants of an impending attack. The levels of such VOCs present in or around planted crops are therefore sometimes identified by collecting samples from the plant environments and subsequently, chemically analyzing the samples in a laboratory using techniques such as gas chromatography and mass spectroscopy. A determination of the relative levels of VOCs can then be made after additional computations and evaluation of the results.

### BRIEF DESCRIPTION OF THE FIGURES

FIG. 1 is a block diagram of an example of a system including real-time condition assessment for living plants according to some aspects of the present disclosure.

FIG. 2 is a flowchart of an example of a process for real-time condition assessment for living plants according to some aspects of the present disclosure.

FIG. 3 is a block diagram of an example of a sensor package for use in real-time condition assessment for living plants according to some aspects of the present disclosure.

FIG. 4 depicts the real part of a measured electrical input impedance for an example of a sensor array used in real-time condition assessment for living plants according to some aspects of the present disclosure.

FIG. 5 is a schematic diagram of an example of a test setup for a sensor array used for real-time condition assessment for living plants according to some aspects of the present disclosure.

FIG. 6 is a data flow diagram showing examples of feature extraction, selection, and classification for real-time condition assessment for living plants according to some aspects of the present disclosure.

FIG. 7 shows graphs of examples of baseline correction and feature extraction for sensor array data according to some aspects of the present disclosure.

FIG. 8 shows graphs of examples of frequency shifts for sensors in an array according to some aspects of the present disclosure.

FIG. 9 shows graphs of examples of frequency changes caused by chemical concentration changes for sensors according to some aspects of the present disclosure.

FIG. 10 shows examples of confusion matrices for gas classification by a machine-learning model for real-time condition assessment for living plants according to some aspects of the present disclosure.

FIG. 11 shows graphs of an example of the response of sensors as affected by humidity level according to some aspects of the present disclosure.

FIG. 12 is an example of a confusion matrix for gases with different levels of added humidity as determined by a machine-learning model for real-time condition assessment for living plants according to some aspects of the present disclosure.

### DETAILED DESCRIPTION

Plant disease presents a crucial challenge to agricultural production, often causing over 30% of crop losses, which can globally affect food security, and consequently pose a significant risk for not only human health, but also the global economy. Agriculture and forestry have been affected adversely by the damage caused by accidental introduction of non-native species. Destructive species such as many weeds, pest insects, and plant pathogens cause several billions of dollars' worth of losses annually in the United States alone. In addition, several billions of dollars are spent on pest and disease management. The major causes of plant diseases include bacterial, fungal, or viral infections, and infestation by insects. It is well known that such pests and diseases, once introduced into an area, have the potential to spread rapidly over large regions if mitigation strategies are not implemented in a timely manner. Treatment for pests and diseases over a larger region can be costly, both financially and environmentally.

Several disease detection techniques for crop protection have been developed over a number of years. These detection techniques can be categorized as direct or indirect. Examples of technologies that have been used for disease diagnosis applications include enzyme-linked immunosorbent assays (ELISA), polymerase chain reaction (PCR), immunofluorescence (IF), flow cytometry (FCM), and fluorescence in-situ hybridization (FISH). These techniques are typically performed in laboratory conditions with complex instruments requiring specific expertise to operate. Sampling processes for the collection and analysis of VOCs can be cumbersome, time-consuming, costly and not scalable or suitable for remote monitoring in the field.

Certain aspects of this disclosure relate to real-time, distributed, remote sensing of volatile organic compounds (VOCs) from plants in order to identify stresses and stages of stress in living plants such as those growing in a field of crops, an orchard, or a greenhouse. A gas sensing platform can include small, low-power, wireless gas sensor packages for selective detection of VOCs released from plants under different conditions, including abiotic or biotic stress conditions. The sensor packages for the platform can be implemented using an array of capacitive micromachined ultrasonic transducer (CMUT) arrays, in which elements are functionalized with a variety of materials including polymers, phthalocyanines, and metal, to improve selectivity. A cloud-based, local, or remote computing platform can receive data from the arrays of sensors. The computing platform can determine a characteristic about a nearby plant or nearby plants based on chemicals detected in the gas emissions from the plants and produce a plant condition assessment based on the characteristic.

Since the relative response of different sensors to tested VOCs can vary due to environmental and other factors, the platform can include a machine-learning model trained for gas classification with specific arrays of sensors. Various sensor arrays with selectivity for detection of VOCs specific to plant species or plant infections of interest can be produced and the machine-learning model can be retrained as necessary to provide accurate gas classification. Relative levels of VOCs as detected in the gas emissions of nearby plants can be treated as a chemical fingerprint and used as the characteristic upon which a condition assessment is based, for example, by matching it to one or more known chemical fingerprints indicating one or more specific plant conditions. This process can take place continuously in real-time or near real-time, to provide early identification of plant stress and its cause, such as a pathogen or trauma. The condition assessment can be produced substantially contemporaneously with the gas emissions. The condition assessment may also include, as further examples, indications that the plants are healthy, that they have adequate or inadequate hydration, or that they have reached a certain stage of development, such as when they are ripe or ready for harvesting.

Plants under stress can emit a wide range of VOCs. For example, one well-known stress VOC is (Z)-3-Hexenol, which is emitted by plants immediately upon tissue damage caused by herbivores. (Z)-3-Hexenol is an alcohol and member of the green leafy volatile (GLV) family, which is synthesized by the lipoxygenase (LOX) pathway. The volatiles associated with the GLV family can be recognized by their distinct fresh cut grass aroma. In the LOX pathway, fatty acids are oxygenated at the 9- or 13-carbon position of linoleic or linoleic acids to produce hydroperoxy fatty acids. The hydroperoxy fatty acids are then transferred into at least seven distinct sub-branches to produce oxylipins. These oxylipins are a diverse group of oxygenated fatty acid metabolites. GLVs produced by stressed plants can be produced by this pathway.

Another group of well-known herbivore-induced plant volatiles are terpenes. Increased terpene emissions have been shown to improve plant defense against herbivores. One well-known example of a terpene compound is linalool. Linalool is a monoterpene alcohol that has a sweet floral scent and is produced by a variety of plants. Linalool has been shown to be emitted during insect herbivory, such as in the case of maize damaged by caterpillars as well as pathogen infection, as in the case of gray mold on strawberry fruits. Another monoterpene of note is 1-Octanol. 1-Octanol is emitted from homogenized wheat meal inoculated with Aspergillus and Penicillium species. 1-Octanol has also been shown to be emitted by potato tubers following infection with Fusarium coeruleum and Phytophthora infestans. Additionally, 1-Octanol produced from watercress leaves has been demonstrated to have nematocidal activity against Meloidogyne incognita.

VOCs are a common means to communicate between organisms. Styrene is an interesting example of this crosstalk that leads to increased plant protection from Bacillus mycoides in the rhizosphere soil of tomato plants. Styrene can exhibit high nematicidal activity against the root-knot nematode M. incognita. Though widely used in industry, styrene does occur naturally, such as in several trees in the styracaceae family and fungi. In fungi, such as Aspergillus, Penicillium, Saccharomyces, and Trichoderma, styrene has been reported to be produced by the non-oxidative decarboxylation of cinnamic acid. Dimethylbenzene, specifically, p-Xylene is another aromatic hydrocarbon and is one of three isomers. P-Xylene is used extensively in industry, however, it can also be produced in plants including both winged beans and soybeans. The bacteria Bacillus amyloliquefaciens and B. thuringiensis are also known to produce p-xylene in the rhizosphere of the Bambara groundnut and it has been shown to have bacterial growth inhibition properties.

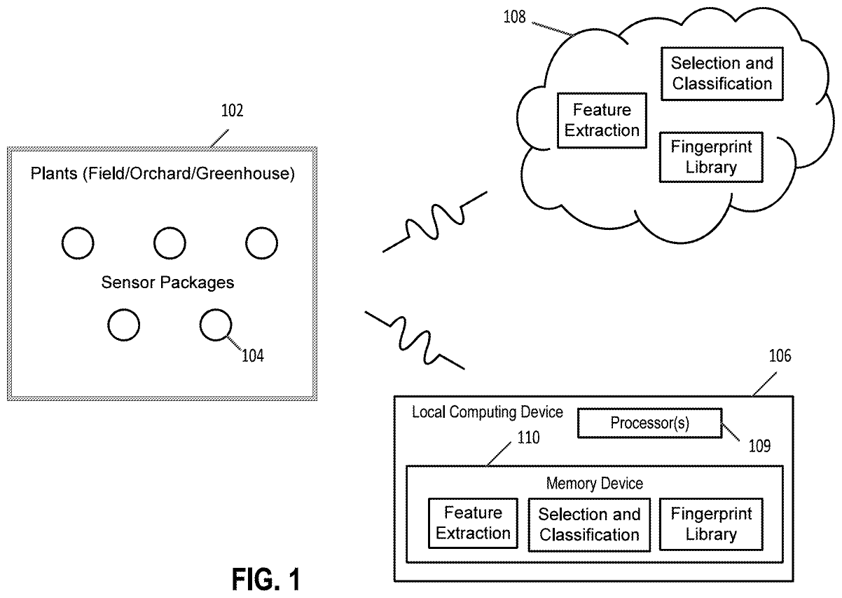

FIG. 1 is a block diagram depicting an example of a system including real-time distributed sensing according to some aspects of the present disclosure. The system of FIG. 1 provides sensing for plants distributed in an area 102. Area 102 can be, as examples, a field, an orchard, or a greenhouse. Sensor packages 104 are distributed throughout area 102. The sensor packages can be small enough to be clipped to plants, installed on small stakes, installed on wires, or distributed in any other way among the living plants in area 102. Each sensor package 104 can include an array of chemical sensors. Each sensor package 104 can also include additional sensors, for example, environmental sensors for humidity, pressure, temperature, etc. The sensor packages can transmit data wirelessly to a computing device 106. Alternatively, or in addition, the sensor packages can transmit data wirelessly to a cloud-based platform 108.

As an example, computing device 106 may be a computing device located near the area 102 where gases in plant emissions are being monitored, for example, in a farmhouse or farm office. Computing device 106 can alternatively be located remotely. Examples of the computing device 106 can include a server, laptop computer, desktop computer, smartphone, tablet computer, or any combination of these. Computing device 106 includes a processor device 109, which may include one or more processors that can execute computer program code, also referred to as software, instructions, or program code instructions for performing operations related to determining, based on the data from the sensor packages 104, a characteristic about the living plants in area 102, and producing a plant condition assessment of the living plant based on the characteristic. Processor device 106 is communicatively coupled to the memory device 110. The memory device includes the computer program instructions for receiving, storing, and processing data from the sensor packages as well as for feature extraction and classification. The memory device also includes a chemical fingerprint library. In some examples, at least some of the memory device 110 can include a non-transitory computer-readable medium from which the processor device 106 can read instructions. A computer-readable medium can include electronic, optical, magnetic, or other storage devices capable of providing the processor device with computer-readable instructions or other program code.

Cloud-based platform 108 can include the same or similar computer program instructions as described above, stored on servers with similar processor devices and memory devices. The use of a cloud-based system can allow the processing necessary to interpret data from the sensor packages to be provided as a service. A stand-alone computing device can still be used with a cloud-based platform to receive plant condition assessments or sensor information, either via a Web browser or an application designed for this purpose.

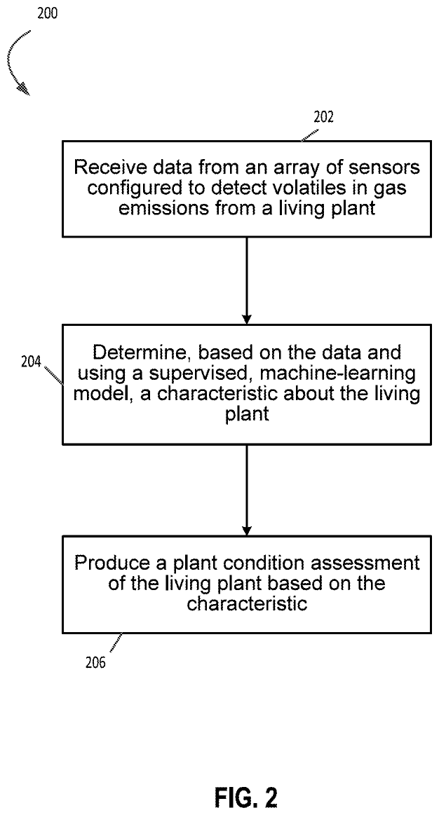

FIG. 2 is a flowchart of an example of a process for real-time condition assessment for living plants according to some aspects of the present disclosure. In some examples, a processor device such as processor 109 can perform one or more of the operations shown in FIG. 2 to provide real-time condition assessment. In other examples, the processor device can implement more operations, fewer operations, different operations, or a different order of the operations depicted in FIG. 2. Process 200 of FIG. 2 is described below with reference to components discussed above.

At block 202, the processor device receives data from an array of sensors configured to detect volatiles in gas emissions from a living plant. For example, the processor device 109 may receive data from an array of sensors in one of the sensor packages 104. At block 204, the processor device can determine, based on the data, and using a supervised, machine-learning model, a characteristic about the living plant. The characteristic may be a chemical fingerprint determined using feature extraction, and using selection and classification. The feature extraction as well as the selection and classification may be implemented by local computing device 106 or cloud computing platform 108. At block 206, the processor device can produce a plant condition assessment of the living plant based on the characteristic.

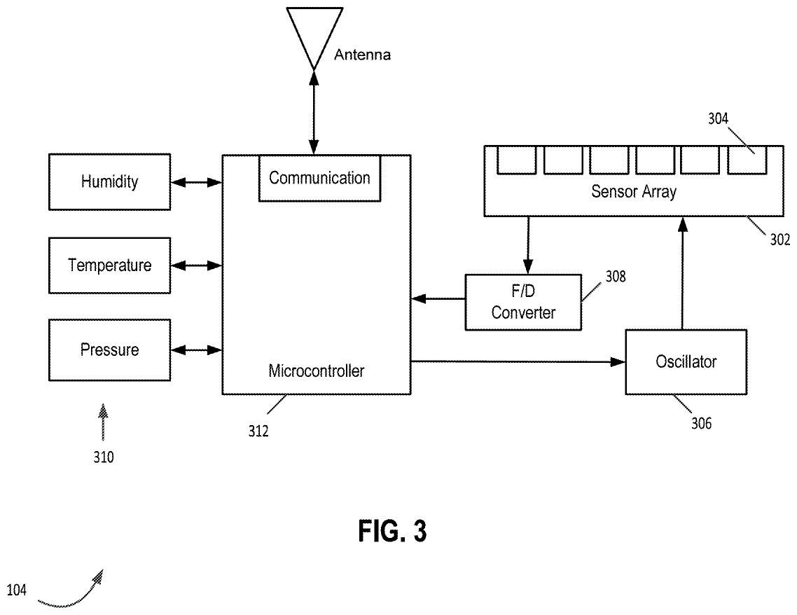

FIG. 3 is a block diagram depicting an example of a sensor package 104 for use in real-time distributed sensing according to some aspects of the present disclosure. The sensor package includes a sensor array 302 of multiple sensors 304. In this example, at least some of the sensors include a capacitive micromachined ultrasonic transducer (CMUT) functionalized with one or more layers of organic or inorganic materials so that the sensor acts as a gravimetric sensor configured to respond to one or more chemicals such as a volatile in gas emissions from living plants. In comparison with other electromechanical resonators, the CMUT resonators offer several advantages for gravimetric sensing. These include the ability to define a multi-element structure on a single die, the resulting multicellular construction of an array, easy integration with electronics, fine mass resolution, and fast response. The CMUT acts as an electrostatically actuated flexural mode microelectromechanical resonator. Microfabrication techniques can be used to fabricate CMUTs on wafers processed in batches. The multicellular structure helps minimize mechanical noise and consequently improves the limit of detection. Multiple elements functionalized with different sensing layers on a single die combined with multivariate signal processing techniques can be used for selective identification of different analytes in gas phase. A custom integrated circuit can be produced that includes an electrical oscillator 306 and a frequency-to-digital (F/D) converter 308, used to track changes in the resonant frequency of each sensor caused by mass changes resulting from adsorption and desorption of gas molecules on the surface of functionalization layers. Pressure, temperature, and humidity sensors 310, either discrete or packaged together, can also be included in a common housing. For example, a BME280 combined sensor package can be acquired from Bosch Sensortec GmbH, Reutlingen, Germany. A microcontroller 312 enabled with a communication interface can provide for control of the operation of the sensor package and to acquired data. In one example, Bluetooth communication can be provided by a Simblee® microcontroller, RF Digital Corp, Hermosa Beach, CA. Power can be supplied through a small battery (not shown) and the sensor array can be bonded in a plastic leaded chip carrier (PLCC) so that it can be conveniently replaced.

### Examples of Functionalization

There are several methods to apply a sensing layer to the surface of a CMUT's resonator depending on the coating material. For example, the surface of a CMUT can be functionalized with polyisobutylene (PIB, Mw˜500 k, Sigma Aldrich, Milwaukee, WI) or copper (II) phthalocyanine (CuPc, Mw=576.08, TCI, Portland, OR) by using a drop-coating method. A surface of a CMUT can be functionalized with silver using ink-jet with silver ink (Ag, Liquid X Printed Metal, Pittsburgh, PA). The surface of CMUT can include a gold layer between the resonator itself and the functionalization layer. One variable for functionalization of the CMUT array is the concentration of the solution which can be chosen to prevent the surfaces of the CMUTs from overloading. A 0.1 wt. % solution has been used to ensure the mechanical loading by the resulting film is minimal after the evaporation of the solvent, and as a result, the oscillation in the readout circuit can be sustained even after the functionalization step. A 0.5-μl droplet of the prepared PIB solution (in Toluene: >99.5%, Sigma Aldrich, Milwaukee, WI) and CuPc (in Chloroform: 99.8%, Acros Organics, Pittsburgh, PA) were dropped on the CMUT surface by using a volume-controlled micropipette (0.5-10 μL, Eppendorf, Hauppauge, NY). The solvents evaporated at room temperature leaving behind a thin coating of the dissolved material.

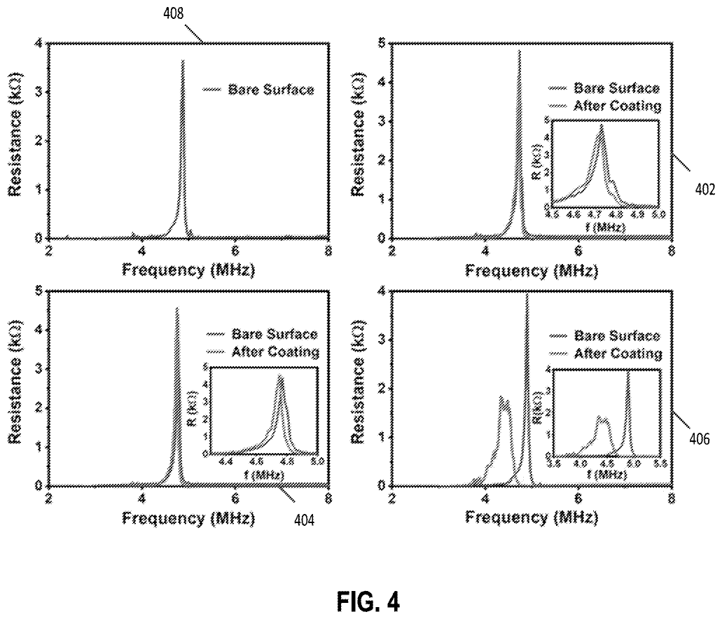

FIG. 4 depicts the real part of a measured electrical input impedance for an example of a sensor array used in real-time condition assessment for living plants according to some aspects of the present disclosure. Ag ink was applied on the CMUT surface with one print pass, and annealed at 150° C. for several minutes to form a thin layer of silver coating. The PIB layers corresponding to graph 402 and CuPc layers corresponding to graph 404 had a uniform and a circular shape while the surface of Ag, corresponding to graph 406 was rough and dispersed resulting in a larger effective surface area, which can improve the sensitivity of the sensor.

To examine the resonant characteristics of the CMUT array elements post-coating for the graphs shown in FIG. 4, the electrical input impedance was measured for each element with a 40-V DC bias. The input impedance pre- and post-coating is compared. Graph 408 represents the result for an un-coated sensor. There was little difference for the PIB and CuPc coatings. Although Ag coating may cause more significant loading, oscillation for this sensor was still sustained when connected to an electrical oscillator indicating that the mechanical loading presented by the coating was acceptable.

### Example of a Test Setup

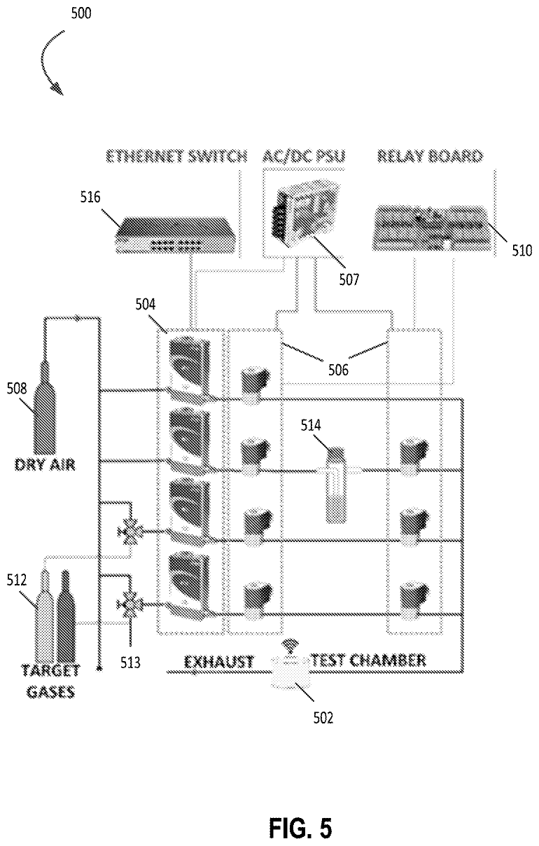

FIG. 5 is a schematic diagram of an example of a test setup for a sensor array used for real-time condition assessment for living plants according to some aspects of the present disclosure. FIG. 5 illustrates a gas-sensing measurement setup 500 that can be used to perform measurements for a CMUT array in a custom-designed Teflon® test chamber 502. The chamber was sealed by an O-ring placed between the open base of the cylindrical chamber and the printed circuit board on which the electronics were implemented as described below with respect to FIG. 5. In the test setup, four (4) mass flow controllers 504 (MFCs, GE50 and GM50 series, MKS Instruments Inc., Andover, MA) and seven (7) two-way solenoid valves 506 (SV121, Omega Engineering Inc., Norwalk, CT) were used to control the flow of the gases connected to each gas line. Power was supplied by power supply 507. A first gas line was dedicated to carrier or dilution gas (dry air 508) and a second gas line was used for generating artificial humidity by bubbling deionized (DI) water in a glass washing bottle. Third and fourth gas lines were used to provide the target analyte gas flow, which was supplied from one or more calibrated gas cylinders 512 or by bubbling the pure liquid form of the analyte using bubbler 514. The source could be selected via a three-way valve 513.

In this example, each mass flow controller had an Ethernet interface connected to switch 516 to allow connectivity via the TCP/IP MODBUS protocol running on the MFC's embedded microcontroller. Solenoid valves 506, which are used to prevent back flow, were connected to a 16-channel USB-controlled relay board 510. All of the described instrument interfaces as well as the Bluetooth link to the CMUT interface electronics were controlled through a graphical user interface (LabVIEW 2018, National Instruments, Austin, TX) running on a personal computer.

As the first operation in the testing procedure, the CMUT array was exposed to dry air flow (200 sccm) to establish a baseline for measuring the relative response to different analytes. Target analytes with appreciable concentrations were then injected into the gas chamber for ten (10) minutes. p-Xylene and Styrene were directly supplied from a calibrated gas cylinder (AirGas Company, Raleigh, NC), and 1-Octanol (98%, Alfa Aesar, Tewksbury, MA), Linalool (97%, Alfa Aesar, Tewksbury, MA) and (Z)-3-Hexenol (98%, Acros Organics, Pittsburgh, PA) were bubbled from pure liquid form at room temperature to provide a saturated vapor pressure calculated using the Antoine equation. The desired concentrations of target analytes were obtained by diluting the analyte flow with the carrier clean air at controlled ratios with the help of the MFCs. The relative humidity (RH) was calculated using the ratio of the flow rate of the second line to the overall flow rate. To change the volume concentration of a target analyte expressed in units of ppm in an ambient RH of 50%, the air flow of the second line was kept at 100 sccm, and then the flow rate of the target analyte connected to the third or fourth gas line was changed to provide an overall flow rate of 200 sccm by also adjusting the flow rate for the diluting portion of the carrier gas.

### Example of Gas Classification Using Machine Learning

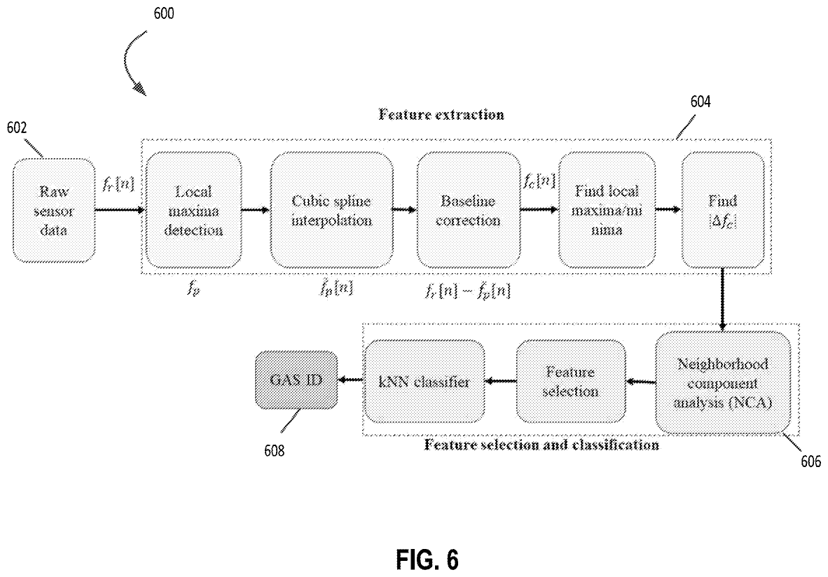

FIG. 6 is a data flow diagram showing examples of feature extraction, selection, and classification for real-time condition assessment for living plants according to some aspects of the present disclosure. For the classification of the gases based on the frequency shifts measured from each sensor channel, a series 600 of data processing operations can be used. Raw sensor data 602 is subject to process 604 for feature extraction. Process 606 provides feature selection and classification to produce a gas identification 608.

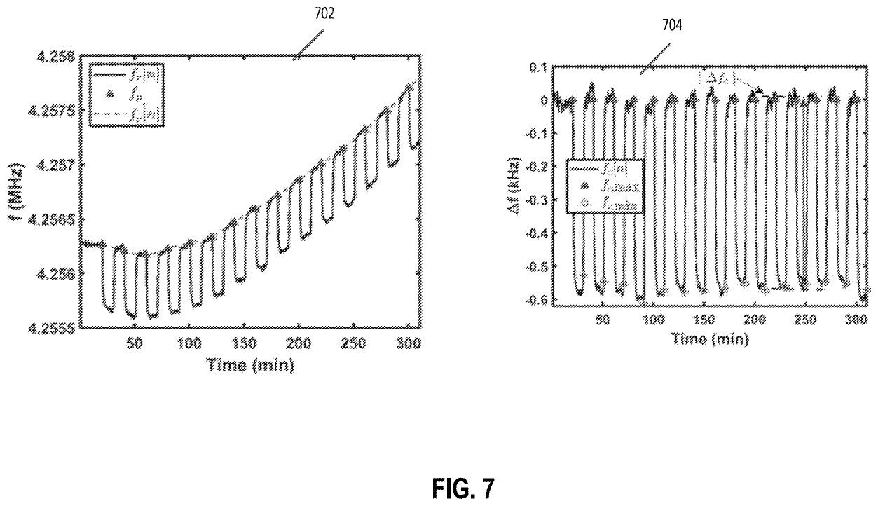

Let f_r be the measured oscillation frequency representing the raw sensor output from a single channel. FIG. 7 shows graphs of examples of baseline correction and feature extraction for sensor array data according to some aspects of the present disclosure. The f_r signal generated by the Ag-functionalized CMUT element in response to a series of ten (10) minute clean air and ten (10) minute 40-ppm p-Xylene pulses is shown in graph 702. In this example, the first operation is to find the frequency shifts between the time when the sensor is exposed to target gas and the time the flow is switched back to clean air. This frequency shift can be used as the single feature in the classification operation of process 606. As the frequency of oscillation for a given sensor channel can drift due to environmental factors, e.g., temperature, that are not related to what the sensor is exposed to as shown in graph 702, the baseline value shifts. To characterize the baseline drift, the local peaks f_p, are denoted by triangular markers. Baseline correction can be performed using cubic spline interpolation between the marked peak points to flatten the baseline before frequency shifts for each gas exposure are extracted. The interpolated curve is denoted by \tilde{f}_p and represented by the red dashed curve in graph 702. Baselined corrected frequency profile f_c can be obtained by subtracting the interpolated signal from the frequency profile of the raw signal, i.e., f_c=f_r-\tilde{f}_p, and shown in graph 704 as part of extraction process 604. Finally, the local maxima and minima in f_c can be determined by differentiation, and the frequency shift | [symbol] Δf [symbol] _c| in each cycle can be calculated, also as part of extraction process 604 to be fed into the classifier.

Following the feature extraction process, a neighborhood component analysis (NCA) can be performed as part of process 606 to determine the impact of each individual sensor and to lower the computational complexity of the classification process. The NCA in this example is a kernel-based feature selection algorithm that helps identify the most descriptive features in a given feature set. Features selected based on the NCA results can be used with the k-nearest-neighbors (kNN) classifier in process 606 to distinguish between the gases. The parameters of the kNN classifier for this particular problem are provided below.

The sensor package can include a filter and air handling capability to enable differential (i.e., filtered clean air sample vs. ambient air sample) measurements. The sample can be gathered from the environment, divided into two parts in the sensor package where one part is filtered and the other part is unfiltered, and one or more of the sensors are then exposed to these two samples to measure the response of the sensor to the actual, unfiltered sample in comparison to the filtered clean-air reference.

### Example of Response of a Functionalized CMUT Array to Different Volatiles without Humidity

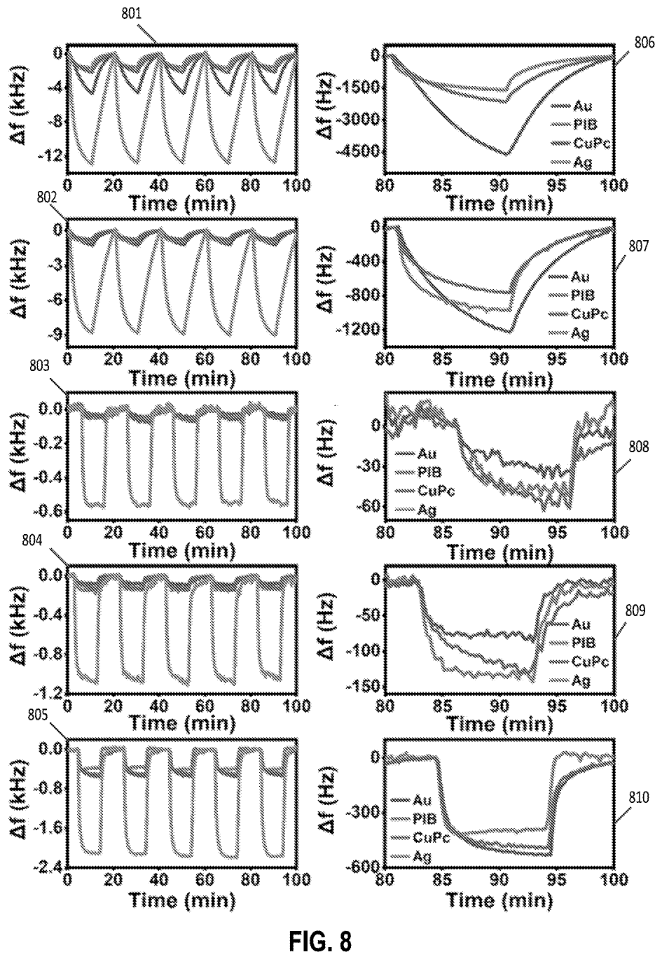

FIG. 8 shows graphs of examples of frequency shifts for sensors in an array according to some aspects of the present disclosure. The gas sensing performance of the functionalized CMUT array was investigated for plant volatiles 1-Octanol, Linalool, p-Xylene, Styrene, and (Z)-3-Hexenol with different relative humidity levels (0, 10, 25, 50%) at room temperature. The performance of the array with dry air flow (0% RH) is first presented and then the performance with added humidity is discussed.

Responses were recorded from all four channels in the array for the listed five plant volatiles at three different concentrations for each, in dry flow conditions. By repeating the exposure for each concentration 15 times, a data set of size of 225 samples was collected for dry conditions. A sample collection of experimental data for approximately 40-ppm concentration of five different VOCs without humidity is shown in graphs 801-810. Frequency shifts as a function of time are shown in graphs 801-805 as follows: 1-Octanol 801; Linalool 802; p-Xylene 803; Styrene 804; and (Z)-3-Hexanol 805. Corresponding responses of the three sensors tested are shown in graphs 801-810, respectively. A protocol was set for the carrier, and the target gases were switched after ten (10) minutes of exposure. Even though in some cases there was no saturation of the response (i.e., the steady state was not reached), e.g., for 1-Octanol and Linalool, in ten (10) minutes, for most of the data set, the steady state was reached in several minutes. Reaching steady state is not required to differentiate gases.

The results show that 1-Octanol created the most significant response for each sensor tested. The Au, PIB, CuPc, and Ag coated sensors exposed to 40-ppm 1-Octanol generated a frequency shift of 4.58, 1.68, 2.16, and 12.98 kHz, respectively. It was also observed that all sensors exhibited a strong response when exposed to gases in the alcohol group, which might be related to the enhanced adsorption properties for (OH)⁻, especially for metal based materials. Since the frequency shifts of the Ag sensor were distinguishably dominant when comparing to the others, the responses recorded from the other channels are also shown separately in graphs 806-810. The Au sensor showed higher responses for 1-Octanol, Linalool, and (Z)-3-Hexenol, while it had a weaker response to the other gases.

### Example of Sensitivity Analysis of a Functionalized CMUT Array without Humidity

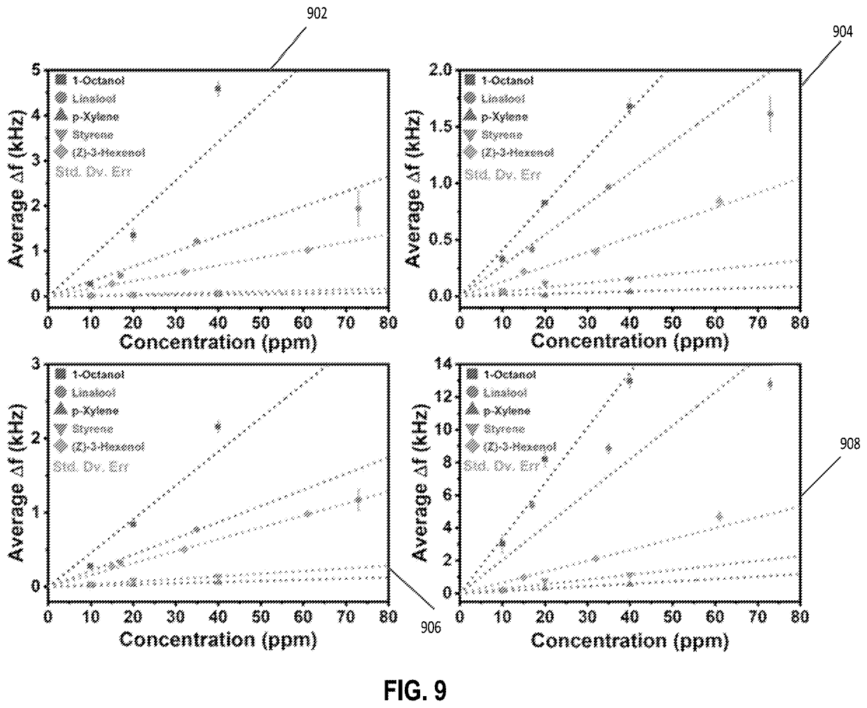

FIG. 9 shows graphs of examples of frequency changes caused by chemical concentration changes for sensors according to some aspects of the present disclosure. The frequency shifts exhibited on four sensor channels for three different concentrations of five VOCs tested were analyzed to determine the sensitivity of the sensors to the target analytes. The average of fifteen frequency shifts acquired consecutively was calculated for each one of the three concentrations used for each of the volatiles and is shown, along with the calculated standard deviation. Graph 902 shows the results for Au, graph 904 shows the results for PIB, graph 906 shows the results for CuPc, and graph 908 shows the results for Ag. The sensitivities of the CMUT sensor channels were defined as the slope of a linear fit from 0 to 80 ppm. The average Δf's increased proportionally to the VOC concentration, and the characteristics were mostly linear in this range of concentrations.

Another performance metric for a gas sensor is the limit of detection (LOD), which defines the lowest detectable concentration of the target gas. To calculate the LOD of each CMUT element tested, the inverse slope of the linear-fitted line in FIG. 9, was used as the frequency resolution of the frequency-to-digital converter in the circuit was 1 Hz when a gate time of 500 ms is used. The sensitivity (Hz/ppm) and the LOD (ppb) data for the four sensor channels are listed in Table I. Based on the 1 Hz frequency resolution, the lowest LOD was calculated as 3 ppb for the Ag-coated CMUT element when exposed to 1-Octanol. The higher gas sensing performance of Ag-coated CMUT element may be for purposes of this example attributed to the larger effective surface area of the Ag layer, which provides more suitable gas adsorption sites by the oxygen containing groups.

TABLE I
Gas sensing performance of the functionalized CMUT sensors.

| | Sensitivity (Hz/ppm) | | | | Limit of Detection (ppb) | | | |
|---|---|---|---|---|---|---|---|---|
| Gas | Au* | PIB | CuPc | Ag | Au* | PIB | CuPc | Ag |
| 1-Octanol | 85 | 41 | 45 | 337 | 12 | 24 | 22 | 3 |
| Linalool | 33 | 27 | 22 | 205 | 30 | 37 | 45 | 5 |
| p-Xylene | 1 | 1 | 1.6 | 15 | 1000 | 909 | 625 | 67 |
| Styrene | 2 | 4 | 3.5 | 29 | 476 | 250 | 286 | 35 |
| (Z)-3-Hexenol | 17 | 13 | 16 | 67 | 59 | 77 | 62 | 15 |

*No coating
Sensitivity and LOD were calculated in a range of 0-80 ppm gas concentration.

It has been known in chemisorption that the electronic densities are arranged with a chemical bond, which is often a thermally activated process. Alternatively, physisorption of molecules involves relatively weak intermolecular forces including dispersion, dipolar or van der Waal interactions between the surface and the gas molecules. In physical sorption, a large redistribution of electron densities for either the surface or the gas molecules does not take place. The overlap of the wave functions of the molecule and the substrate is rather small, and no major change in the electronic structure is usually observed. Moreover, an adsorbed molecule may wander along the surface of a metal because there is no true chemical bond between physisorbed species and the surface. This mobility of physisorbed molecules differs for many metallic surfaces due to differences of their catalytic properties. In some cases, π-interaction using delocalized π-electron in an aromatic ring can provide an explanation of the interaction between VOCs and the metal surface. The surface morphology, like roughness or porosity, can play a significant role in the interaction with the gas molecule, and the sensitivity as well.

The surface interactions for metal-phthalocyanines are mainly determined by van der Waal bond energies between the polarizable aromatic rings of the phthalocyanine and the gas molecules. Specific interactions involving electronic states of the central metal may play only a minor role. However, the strong sensitivity of the metal-free phthalocyanine to alcohols with the hydroxyl group (OH) is strongly suppressed if the phthalocyanine ring contains a central metal atom. In phthalocyanine-based on chemiresistor gas sensors, the charge carriers in valance/conduction band change as a result of the adsorption of the gas analyte, which can provide the main sensing mechanism for the chemiresistive sensors.

For a polymer-based chemiresistive gas sensor, the sensing properties can be explained by polymer swelling during interaction with VOCs, thus changing the resistance of the materials. In physisorption, the interaction is weak, essentially maintained by van der Waal forces. These forces are generated by the formation of temporary dipoles due to the polarization of nearby particles. PIB is known as a low-polarity polymer and has considerably less affinity to polar molecules. It has been reported that the hydrogen atoms on the aromatic nucleus of the toluene molecule have a small dipole whereas the octane is a nonpolar molecule. Moreover, for the rubbery polymers like PIB, organic solvent vapors can be absorbed by a dissolution process.

### Example of Classification of Plant Volatiles at Low Concentrations with no Added Humidity

A kNN model with k-fold cross validation (with k=10) can be used for the gas classification task. The dataset consisting of 225 samples (45 samples for each of the five (5) gas classes) were grouped into ten (10) subsets, and each time, one of the ten (10) subsets was used as the test set, and the remaining subsets were used for training the model. The hyper parameters of the kNN classifier (i.e., number of neighbors and distance metric) are optimized using the Bayesian optimization method. For the dataset corresponding to the test described above, the optimum number of neighbors was calculated as three (3), and the optimum distance metric was the Mahalanobis distance.

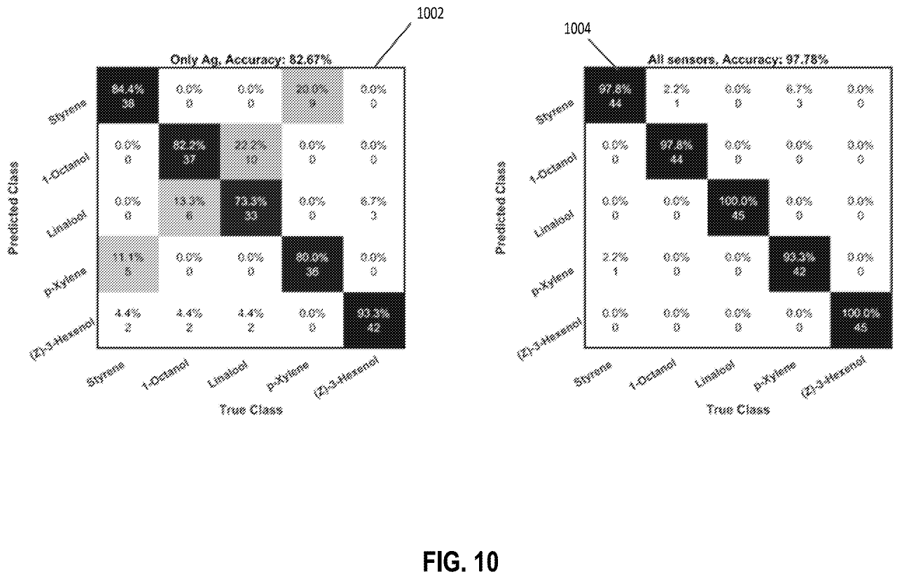

FIG. 10 shows examples of confusion matrices for gas classification by a machine-learning model for real-time condition assessment for living plants according to some aspects of the present disclosure. The confusion matrix 1002 obtained for the five (5) gases in the above-described testing at low concentrations with no added humidity is shown for the Ag sensor. When only the features that were extracted from the Ag-coated sensor channel were used, classification accuracy was found to be 82.67%. The diagonal entries in the confusion matrix 1002 show the number of correct predictions and the corresponding classification accuracy (in percent) for each gas; whereas, off-diagonal elements show the number of mislabeled samples from each class. It was observed that 186 samples out of 225 samples are classified correctly, and that most of the confusions are between p-Xylene and Styrene gases, and Linalool and 1-Octanol gases.

The confusion matrix 1004 is for the same dataset when features from all sensor channels in the test were used is shown in FIG. 10 as well. It is observed that all the samples from (Z)-3-Hexenol and linalool were classified correctly. All the confusions between the Linalool and the 1-Octanol gases were cleared. The confusions observed between p-Xylene and Styrene gases might be related to the similarities in the molecular structure due to the presence of the benzene ring. Although there were a few newly introduced confusions, overall classification accuracy increased to 97.78% with the use of the features from other three (3) sensors, and 220 samples were correctly classified out of 225 cases.

### Example of Gas Sensing Performance of a Functionalized CMUT Array with Added Humidity

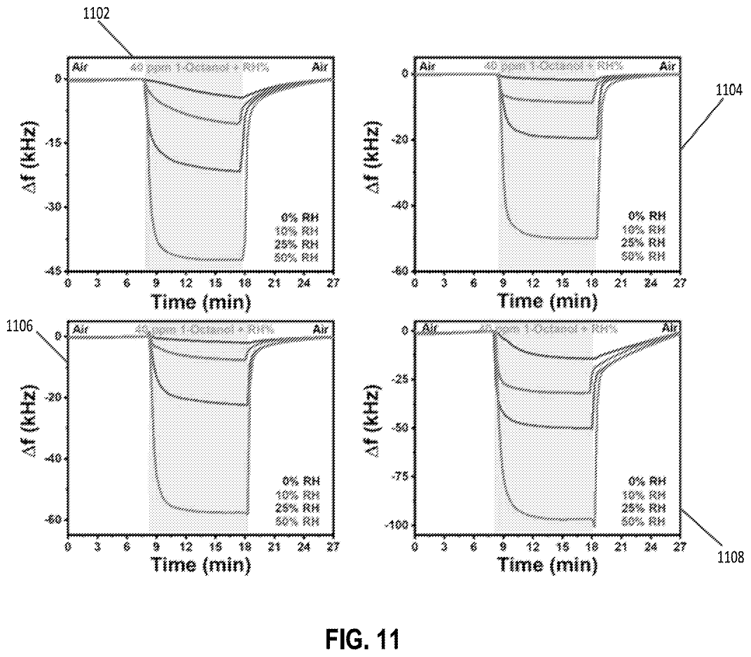

FIG. 11 shows graphs of an example of the response of sensors as affected by humidity level according to some aspects of the present disclosure. As an example, to demonstrate the effect of added humidity on the sensor output, the frequency shift for all four (4) sensor channels in response to 40-ppm 1-Octanol was measured at different humidity levels (0, 10, 25, 50% RH). Graph 1102 shows the response for Au, graph 1104 shows the response for PIB, graph 1106 shows the response for CuPc, and graph 1108 shows the response for Ag. It can be seen that the frequency shifts of all channels in the CMUT array tested increase with the increasing relative humidity level. Humidity has an adverse effect on the sensitivity for some types of sensors, especially metal oxide chemiresistors. The adsorption of water molecules leads to less chemisorption of oxygen species on the metal oxide surface because of the reduction in the available surface area, which results in a weaker sensor response. Furthermore, long-term exposure to humid environments causes debasing of the sensitivity of metal-oxide gas sensors unless they are heated to high temperatures (typically about 400° C.) to help surface hydroxyls become desorbed. Unlike metal-oxide sensors, gravimetric sensors typically do not need to be reset by high-temperature cycling or exposure to UV to desorb the water molecules, because physisorption of the water molecules can be reversed simply by purging the surface with clean air. Furthermore, with the sensors described and tested in this example, the different absorption rates of water molecules on the functionalization layers on the surface of the CMUT array elements can contribute to the pattern recognition and hence enhance the accuracy of the classification of the VOCs even across differing humidity environments.

Stronger sensor response at higher humidity levels can be attributed to a hydroxyl group of water molecules first interacted with the functionalization layer. The next H₂O molecules are then adsorbed onto the hydroxyl layer by hydrogen bonding to form a first H₂O layer and consequently, new H₂O molecules are physically adsorbed onto the previous H₂O layer, and so on. Moreover, 50% RH is equal to approximately 14,000-ppm volume concentration of water molecules in air, which is considerably higher than the concentration of plant volatiles in reported measurements.

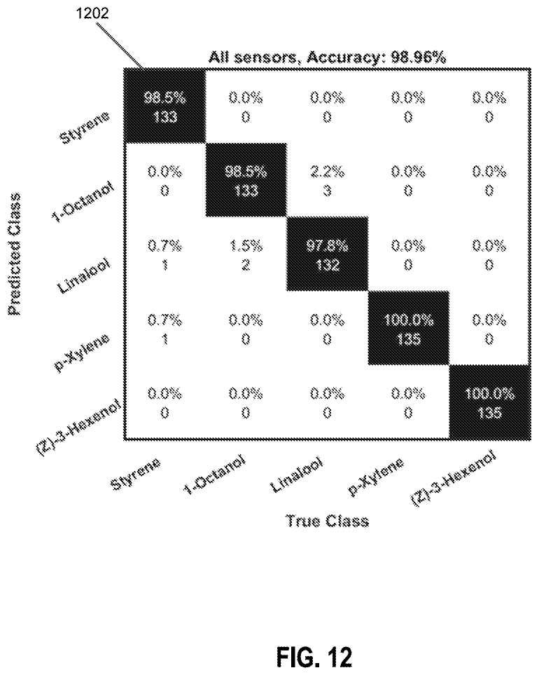

FIG. 12 is an example of a confusion matrix for gases with different levels of added humidity as determined by a machine-learning model for real-time condition assessment for living plants according to some aspects of the present disclosure. For classifying target analytes with different levels of added humidity, a dataset with 675 samples has been tested. The included variations in the dataset include three (3) concentrations of five (5) target analytes tested at three (3) relative humidity levels. At each experimental condition frequency shift data was collected for 15 ten (10) minute pulses of gas exposure resulting in a total of 675 samples with 135 samples per class. FIG. 12 shows the confusion matrix 1202 produced as result of the kNN classification. 668 cases were correctly classified out of the total 675 cases. As compared to the previous classification results for the dataset without humidity, the accuracy was found to have increased for the dataset with humidity. An ability to discriminate five (5) target analytes was demonstrated with an accuracy of 98.96% regardless of the humidity level. This result suggests that with training data acquired in a wide range of environmental conditions, a system can differentiate target analytes without the need for a direct measurement of environmental conditions. Alternatively, environmental data, i.e., temperature, humidity, pressure, that are available from the sensor package can be used as additional features for classification.

The foregoing description of the examples, including illustrated examples, of the subject matter has been presented only for the purpose of illustration and description and is not intended to be exhaustive or to limit the subject matter to the precise forms disclosed. Numerous modifications, adaptations, and uses thereof will be apparent to those skilled in the art without departing from the scope of this subject matter. The illustrative examples described above are given to introduce the reader to the general subject matter discussed here and are not intended to limit the scope of the disclosed concepts.

1. A sensor comprising:
an electromechanical resonator; and
a material on the electromechanical resonator such that the electromechanical resonator is configured to respond to a chemical in gas emissions from a living plant.

2. The sensor of claim 1, wherein the sensor is in an array of sensors of a system, the array of sensors being configured to detect the chemical in gas emissions from the living plant, the system further including a computing device comprising:
a processor device;
a non-transitory computer-readable medium with instructions executable by the processor device to cause the computing device to perform operations, the operations comprising:
receiving data about the chemical from the array of sensors;
applying a supervised, machine-learning model to the data to determine a characteristic about the living plant; and
producing, substantially contemporaneously with the gas emissions, a plant condition assessment of the living plant based on the characteristic.

3. The sensor of claim 2, wherein the non-transitory computer-readable medium includes further instructions executable by the processor device to cause the computing device to perform operations to generate the supervised, machine-learning model by:
receiving a dataset including a plurality of samples corresponding to the array of sensors;
training a k-nearest-neighbor (kNN) model using the dataset; and
optimizing hyper parameters for the kNN model.

4. The sensor of claim 2, wherein the system comprises a common housing including the array of sensors and at least one of a humidity sensor, a temperature sensor, or a pressure sensor.

5. The sensor of claim 4, wherein the data includes a measurement from the at least one of the humidity sensor, the temperature sensor, or the pressure sensor to provide features for the supervised, machine-learning model to determine the characteristic about the living plant.

6. The sensor of claim 2, wherein the array of sensors is disposed in a batch-fabricated, multicellular structure.

7. The sensor of claim 4, wherein the material comprises at least one of an organic or an inorganic gas-sensitive layer.

8. The sensor of claim 4, wherein the common housing further comprises a filter to provide a clean-air reference for comparison to an unfiltered sample.

9. A method comprising:
receiving, by a processor device, data from an array of sensors configured to detect volatiles in gas emissions from a living plant;
determining, by the processor device, based on the data and using a supervised, machine-learning model, a characteristic about the living plant; and
producing, by the processor device, a plant condition assessment of the living plant based on the characteristic.

10. The method of claim 9, further comprising:
receiving a dataset including a plurality of samples corresponding to the array of sensors;
training a k-nearest-neighbor (kNN) model using the dataset; and
optimizing hyper parameters for the kNN model to generate the supervised, machine-learning model.

11. The method of claim 9, further comprising:
receiving a measurement from at least one of a humidity sensor, a temperature sensor, or a pressure sensor; and
using the measurement to provide a feature for determining the characteristic about the living plant.

12. The method of claim 9, further comprising depositing a material on an electromechanical resonator to form at least one sensor in the array of sensors such that the electromechanical resonator is configured to respond to at least one of the volatiles in the gas emissions from the living plant.

13. The method of claim 12, wherein the material comprises at least one of an organic or an inorganic gas-sensitive layer.

14. The method of claim 9, further comprising dividing an air sample into a filtered, clean-air reference and an unfiltered sample, and wherein determining the characteristic about the living plant further comprises determining a response of the array of sensors for each of the clean-air reference and the unfiltered sample.

15. A system for providing a plant condition assessment for a living plant, the system comprising:
an array of sensors positionable near the living plant configured to be responsive to chemicals in gas emissions from a living plant;
a computing device configured to receive data from the array of sensors; and
at least one memory device including instructions that are executable by the computing device for causing the computing device to perform operations comprising:
determining, based on the data and using a supervised, machine-learning model, a characteristic about the living plant; and
producing the plant condition assessment of the living plant based on the characteristic.

16. The system of claim 15, wherein at least one sensor in the array of sensors comprises:
an electromechanical resonator; and
a material on the electromechanical resonator such that the electromechanical resonator is configured to respond to at least one of the chemicals in the gas emissions from the living plant.

17. The system of claim 16, further comprising at least one of a humidity sensor, a temperature sensor, or a pressure sensor to provide a feature for determining the characteristic about the living plant.

18. The system of claim 17, further comprising a common housing including the array of sensors and the at least one of a humidity sensor, a temperature sensor, or a pressure sensor.

19. The system of claim 18, wherein the array of sensors is disposed in a batch-fabricated, multicellular structure.

20. The system of claim 15, wherein the operations further comprise:
receiving a dataset including a plurality of samples corresponding to the array of sensors;
training a k-nearest-neighbor (kNN) model using the dataset; and
optimizing hyper parameters for the kNN model to generate the supervised, machine-learning model.
